SEQUENCE LISTING (6 sequences)

SEQ ID NO: 1 - protein, 192 aa, Artificial sequence
  Synthetic Construct - Topological structure Linear
MQLTKGRLHFSHPLSHTKHISPFVTDAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 2 - protein, 192 aa, Artificial sequence
  Synthetic Construct - Topological structure Linear
METSQEHQGPSDIHRIPWSWGQRNSHAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 3 - protein, 251 aa, Artificial sequence
  Synthetic Construct - Topological structure Linear
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 4 - protein, 232 aa, Artificial sequence
  Synthetic Construct - Topological structure Linear
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 424 aa, Artificial sequence
  Synthetic Construct - Topological structure Linear
MQLTKGRLHFSHPLSHTKHISPFVTDAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 1338 bp, Artificial sequence
  Synthetic Construct - Topological structure Linear
atggagacagacacactcctgctatgggtactgctgctctgggttccaggttccactggtgacatgcagcttacaaaggggagacttcattttagtcacccactgtcccatacaaaacatatctcacctttcgttaccgacgctccactgcgtgccgacggtgacaaaccccgagcacatctgaccgtggtcaggcagacacccacccagcactttaagaaccagtttcctgctttgcactgggaacacgagctgggcctcgccttcactaagaatcggatgaactacaccaacaaattcctcctgatcccagagagcggagattactttatctacagccaagtcaccttcagaggaatgaccagcgaatgctcagagatacgccaagctgggaggcccaacaaacctgactccattactgtggttattaccaaggtcactgacagttatcccgagcctacacagcttctcatgggcacaaagtccgtatgtgaagtgggatctaattggttccagcccatttatttgggtgccatgtttagtctccaggaaggcgataaactgatggtgaatgtgtctgatatctctttggtggattataccaaggaggataagactttcttcggcgcatttctgctggagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

(19) United States
(12) Patent Application Publication
(10) Pub. No.: US 2013/0211051 A1
(43) Pub. Date: Aug. 15, 2013

(54) FUSION PROTEIN CONTAINING VEGI, AND PHARMACEUTICAL COMPOSITION AND USE THEREOF

(71) Applicants: Xiangming Sun, Shanghai (CN); Ling Zhou, Shanghai (CN); Jiannan Hong, Shanghai (CN); Yun Wu, Shanghai (CN); Jun Bao, Shanghai (CN)

(72) Inventors: Xiangming Sun, Shanghai (CN); Ling Zhou, Shanghai (CN); Jiannan Hong, Shanghai (CN); Yun Wu, Shanghai (CN); Jun Bao, Shanghai (CN)

(73) Assignee: SHANGHAI KEXIN BIOTECH CO., LTD., Shanghai (CN)

(21) Appl. No.: 13/878,495

(22) PCT Filed: Dec. 27, 2010
(86) PCT No.: PCT/CN10/02166

§ 371 Date: Apr. 9, 2013

(30) Foreign Application Priority Data
Oct. 27, 2010 (CN) 201010536456.1

(51) Int. Cl. C07K 14/435; C07K 16/18

(52) U.S. Cl. CPC C07K 14/435; C07K 16/18
USPC 530/387.3

(57) ABSTRACT

Provided is an anti-angiogenic fusion protein, which comprises a vascular endothelial cell growth inhibitor (VEGI) or variant thereof, and other polypeptide such as IgG Fc. The fusion protein optionally comprises a linker. The fusion protein can induce the apoptosis of endothelial cells and inhibit the growtth of endothelial cells, so as to be used for treating tumor. Also provided are a pharmaceutical composition and use of the fusion protein.

### BACKGROUND OF THE PRESENT INVENTION

1. Field of Invention

The present invention relates to the biological medicine field, specifically to a biotechnology of preparation of a fusion protein, and more specifically to an anti-angiogenesis fusion protein, and a pharmaceutical composition and use thereof.

2. Description of Related Arts

The vascular endothelial cell growth inhibitor (VEGI) specifically acts on vascular endothelial cells, and can induce apoptosis of vascular endothelial cells in a growing period and maintain a stationary state of vascular endothelial cells in a stationary period. Presently, four VEGI isomers, VEGI174 of 174 amino acids, VEGI192A and VEGI19213 of 192 amino acids in two different forms have been reported, and VEGI251 of 251 amino acids are found later. These VEGIs have different amino terminals, but have the same core sequence consists of 151 amino acids. All the isomers are derived from the same gene, and are formed through different splicing manners. With human VEGI cDNA as a probe, RNA hybridization is performed on human mRNAs from different cells and tissues. The results show that VEGI is merely expressed in endothelial cells of umbilical vein, aorta and skin capillary vessels. The expression of VEGI is closely related to the growth state of the endothelial cells, the expression of VEGI in endothelial cells in a proliferation state is relatively low, while the expression of VEGI in endothelial cells whose growth has reached contact inhibition is significantly improved, and is several times of that in cells in the growing state. Presently, researches on functions of VEGI are mainly performed on VEGI174.

VEGI174 is a typical type II transmembrane protein, in which 29 to 174 amino acid residues form an extracellular chain, full-length VEGI174 expressed on a cancer cell surface has no influence on growth of tumor, When being expressed in endothelial cells, VEGI 174 has no inhibition effect on growth of the endothelial cells. In researches of other members (such as TNF and FAS ligands) of the TNF family, scientists find that, the TNF and FAS ligands can be cleaved from a membrane and exist in the free form, and can exert their functions. Similarly, artificial recombinant soluble VEGI174 (S VEGI, containing the extracellular chain of VEGI174 and a signal peptide from another secreted protein), when over-expression in tumor cells occurs, the growth of tumor can be inhibited. This indicates that, the full-length VEGI174 has no influence on growth of tumor, while the soluble VEGI can inhibit the growth of tumor. The soluble VEGI174 also inhibits proliferation of bovine aortic and human venous endothelial cells, and the IC50s are respectively 6 ng/ml and 60 ng/ml. However, VEGI of 100 ng/ml has no influence on proliferation of human T cells and bone marrow stromal cells. VEGI192A has stronger inhibition on endothelial cells, while the IC50 of inhibition of growth of bovine aortic endothelial cells is merely 0.272. VEGI192B, VEG 2511, VEGI 25111 and VEGI 2511V have no inhibition effect on growth of bovine aortic endothelial cells, the inhibition effect of VEG 251111 is similar to that of VEGI 174, and the 1050 is 10 ng/ml. VEGI251 is the most abundant VEGI isomer, and contains an inferred secretory signal peptide. Over-expression of secretory VEGI251 leads to apoptosis and growth inhibition of endothelial cells. Similarly, it is also proved that secretory VEGI containing a core sequence of 151 amino acids may start apoptosis and growth inhibition of tumor cells, but the capacity of secretory VEGI is relatively low. In sum, VEGI is not only specifically expressed in endothelial cells, but also specifically inhibits proliferation of endothelial cells, while differences in activity of different variants exist, but the reason for the differences is not clear.

In a model of angiogenesis in vitro, recombinant human VEGI can significantly inhibit formation of tubular structures in collagenous fibers of bovine aortic endothelial cells, and the 1050 is approximately 30 ng/ml. In a chick embryo chorioallantoic membrane angiogenesis in vivo experiment, VEGI may also dose-dependently inhibit capillary vessel formation induced by FGF or VEGF. It can be seen that, regardless of the factor that stimulates angiogenesis, VEGI can inhibit the angiogenesis.

The anti-tumor effect of VEGI is also proved by experiments. Soluble human VEGI is transfected into murine colon carcinoma cells MC-38, and the transfected tumor cells are subcutaneously injected into syngeneic C57BL/6 mouse. The results show that the volume of tumors formed by injected MC-38 cells expressing soluble VEGI is obviously smaller than that of the control group, no adverse reaction is found, and no weight loss is observed. It is interesting that expression of VEGI does not inhibit the proliferation of colon cancer cells, indicating that VEGI has no direct cytotoxicity on tumor cells. It is indicated by immunohistochemical analysis that capillary vessels in tumor are greatly reduced. However, no aggregation of neutrophili granulocyte by VEGI and no infiltration of tumor cells by macrophages. The CHO cells expressing soluble VEGI and human breast cancer cells MADAMB231 are mixed and injected into nude mouse, and it is found that the growth of xenografted tumor is significantly inhibited. These researches show that transfection of human tumor cells with soluble VEGI can inhibit angiogenesis in tumor, and the anti-tumor effect of VEGI is mainly to inhibit angiogenesis.

Many researches show that soluble VEGI merely selectively inhibit growth of vascular endothelial cells, and has no direct toxic effects on other cells such as T cells, B cells and caner cells. However, some researches show that soluble VEGI can directly inhibit growth of four types of cancer cells U-937, MCF-7, Hela, and ML-1 a, especially when a protein synthesis inhibitor cyclohexanone is added, the cytotoxicity get more obvious. In terms of cell-mediated immunity, it is found recently that VEGI192A can induce maturation of dendritic cells, indicating that VEGI has anti-cancer effect, besides inhibition on angiogenesis, stimulation of adaptive immunity of dendritic cells may have anti-tumor effect. These researches show that VEGI may be multi-functional cell factor, and other mechanism of action may exists, in addition to blocking angiogenesis in tumor. In any case, it is clearly proved by a great number of cell and animal experiments that, VEGI has significant anti-tumor effect, and has a promising prospect in clinic applications. Among variants of VEGI, VEGI 192A has the strongest effect.

However, the VEGI molecules have poor stability and are difficult to be expressed, so it is difficult to develop a drug with VEGI for application in clinic anti-angiogenesis treatment.

### SUMMARY OF THE PRESENT INVENTION

In view of the technical problem that existing VEGI molecules have poor stability and are difficult to be expressed, so it is difficult to develop a drug with VEGI for application in clinic anti-angiogenesis treatment, a first technical problem to be solved by the present invention is to provide a fusion protein capable of inhibiting angiogenesis. The fusion protein can fuse VEGI with another polypeptide molecule, so as to increase the stability of the protein, prolong the actuation duration in vivo, and improve the production and preparation of VEGI.

The anti-angiogenesis fusion protein of the present invention is formed by fusing a vascular endothelial cell growth inhibitor and a variant thereof P1 with any other polypeptide P2. The fusion protein has a structural form of P1-P2 or P2-P1.

Furthermore, the anti-angiogenesis fusion protein of the present invention further includes a linker peptide. The fusion protein containing a linker peptide has a structural form of P1-L-P2, P2-L-P1 or P1-L-P1-L-P1.

The vascular endothelial cell growth inhibitor and the variant thereof P1 is VEGI192A or a mutant thereof, and the VEGI192A or the mutant thereof has a homology with the sequence SEQ ID NO: 1 in the sequence list of 80% and more. Or, the vascular endothelial cell growth inhibitor and the variant thereof P1 is VEGI192B or a mutant thereof, and the VEGI192B or the mutant thereof has a homology with the sequence SEQ ID NO: 2 in the sequence list of 80% and more. Or, the vascular endothelial cell growth inhibitor and the variant thereof P1 is VEGI251 or a segment of VEGI251 and a mutant thereof, and the VEGI251 and the segment of VEGI251 or the mutant thereof have a homology with the sequence SEQ ID NO: 2 in the sequence list of 80% and more.

The any other polypeptide P2 of the present invention is human IgG 1 Fe or a mutant thereof, and the human IgG 1 Fc or the mutant thereof has a homology with the sequence SEQ ID NO: 4 in the sequence list of 80% and more.

The linker peptide L of the present invention is (Gly₄Ser)₃.

A second technical problem to be solved by the present invention is to provide an anti-angiogenesis drug with the anti-angiogenesis fusion protein of the present invention which is used as active ingredient, especially an anti-tumor drug.

A third technical problem to be solved by the present invention is to provide a use of the anti-angiogenesis fusion protein of the present invention in preparation of an anti-angiogenesis drug.

In the drug of the present invention, the anti-angiogenesis fusion protein of the present invention is used as the active ingredient, and the drug is used to inhibit angiogenesis, and especially to treat tumor.

Optionally, the drug may contain one or more pharmaceutically acceptable carrier. The carrier includes a conventional excipient, diluent, filler, adhesive, wetting agent, disintegrating agent, absorption enhancer, surfactant, adsorption carrier, lubricant, in the pharmaceutical field. If necessary, a flavor and a sweetener, etc., may be added.

The drug of the present invention may be prepared into an injection preparation for intravenous injection, a percutaneous absorption preparation for subcutaneous injection and external application on skin, nebulas for spraying on nose, throat, oral cavity, skin, and mucous membrane, drops for dropping nose, eye, and ear, suppository for applying to anorectum, a tablet, powder, granule, capsule, oral liquid, paste, cream and other forms. Drugs in different dosage forms can be prepared according to conventional methods in the field of pharmaceutical preparation.

The dose of the drug of the present invention may be adjusted according to the age and weight of a patient and the severity of the disease, and the daily dose generally is 2 to 1000 μg/kg.

The fusion protein of the present invention fuses VEGI with another polypeptide molecule, thereby increasing the stability of the protein, prolonging the actuation duration in vivo, and improving the production and preparation of VEGI. All the components of the fusion protein VF of the present invention are from human source proteins, so no immunogenicity of foreign protein occurs when this protein enters human body as a drug. A VEFI portion of the fusion protein may exert the anti-angiogenesis function of VEGI, and the polypeptide fused with the VEFI portion functions to stabilize VEGI, thereby promoting the expression of VEGI. The fusion protein VF of the present invention blocks angiogenesis in tumor and achieves the purpose of treatment of tumor.

### BRIEF DESCRIPTION OF THE DRAWINGS

FIG. 1 is schematic structural view of a fusion protein VF1 according embodiment 1 of the present invention.

FIG. 2 is a SDS-PAGE electrophoretogram of the fusion protein VF1 obtained through separation and purification according to embodiment 1 of the present invention.

FIG. 3 is a schematic diagram of growth of bovine aortic endothelial cells inhibited by the fusion protein VF1 according to embodiment 1 of the present invention.

FIG. 4 is a schematic diagram of tumor inhibition effect of the fusion protein VF1 according to embodiment 1 of the present invention.

### DETAILED DESCRIPTION OF THE PREFERRED EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The present invention is further described below with reference to embodiments. It should be understood that, the embodiments are merely intended to illustrate, but not to limit the present invention. Any variations made by persons of ordinary skill in the art in implementing the present invention under the teaching of the specification will fall within the scope of the accompanying claims.

### Embodiment 1

### Fusion Protein VF1 and Expression

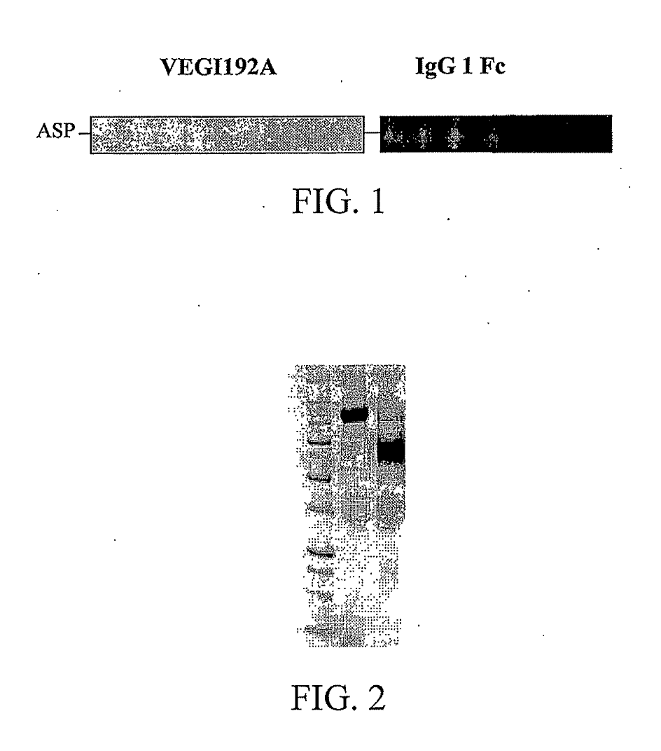

The structure of the fusion protein VF1 of this embodiment is shown in FIG. 1. The structural form is ASP-P1-P2, where ASP is a single amino acid aspartic acid remained after cleaving a secretory signal peptide; P1 is VELI192A, having homology with the sequence SEQ ID NO: 1 in the sequence list of 80% and more; P2 is human IgG 1 Fc, having a homology with the sequence SEQ ID NO: 4 in the sequence list of 80% and more; and fusion protein VF1 is a complete amino acid sequence SEQ ID NO: 5 in the sequence list.

In this embodiment, mammalian cells (CHO cells) are used to express fusion protein VF1. The coding gene sequence used is an amino acid sequence of a protein SEQ ID NO: 6 in the sequence list.

The expression process specifically includes steps of: synthesizing a VF1 coding gene (SEQ ID NO: 6) by an entrusted technical service corporation, and inserting the VF1 coding gene into an expression vector pIRES; amplifying the expression vector by using Escherichia coil, and extracting expression vector of VF1; by using an electroporation method, transfecting the expression vector of VF1 into CHO cells; by using G418, screening out positive clones; and then, culturing recombinant CHO cells in mass, and harvesting the supernatant of the cell culture, separating and purifying Protein A, to obtain fusion protein VF1, as shown in FIG. 2.

### Embodiment 2

### Inhibition on Growth of Vascular Endothelial Cells of Fusion Protein VF1

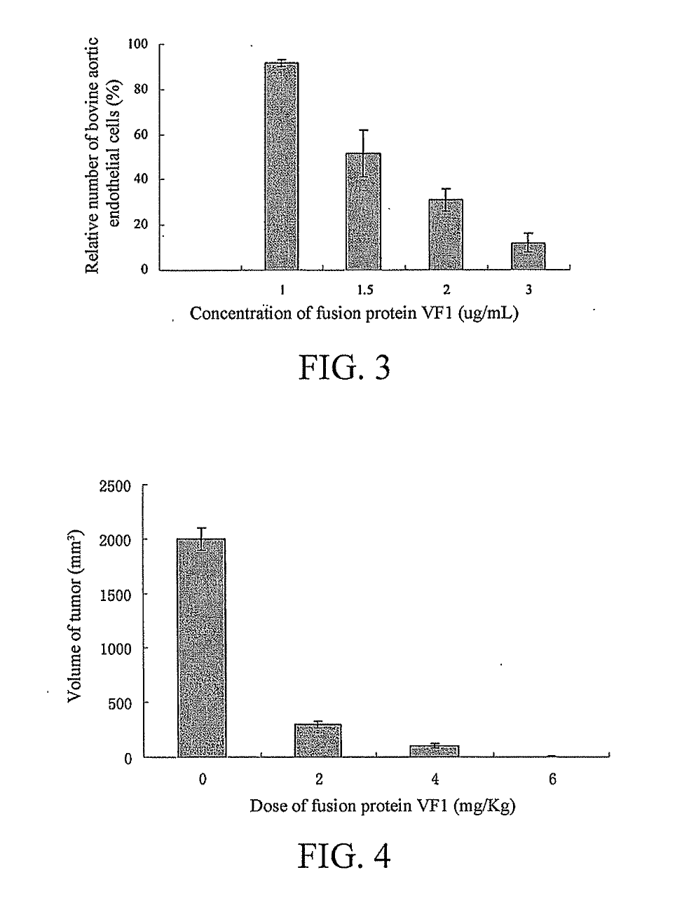

The fusion protein VF 1 obtained in Embodiment 1 was added to bovine aortic endothelial cells at different concentrations, and a clinic buffer was used as control. After 3-day culture, the cells were digested, and the cell density is counted. With the percentage ratio of the cell density to the cell density of the control group as the vertical coordinate, and the concentration of the fusion protein VF as the horizontal ordinate, results were shown in FIG. 3.

This embodiment shows that, the fusion protein VF1 can significantly inhibit growth of bovine aortic endothelial cells.

### Embodiment 3

### Inhibition on Growth of Tumor of Fusion Protein VF 1

Lewis lung cancer cells were cultured in a medium containing 10% fetal bovine serum. Once confluence, the cells were digested by a 0.05% solution of trypsin, and were centrifuged and washed one time with a phosphate buffer, and then re-suspended in a phosphate buffer. 20 C57BL/6 mouse were subcutaneously injected with 2.5×10⁵ Lewis lung cancer cells at the abdomen. 6 days later, the C57BL/6 mouse had subcutaneous tumors formed, and the volume of tumor was up to 100 to 200 mm³, accounting for 0.5% to 1% of the weight. Then, the mouse were divided into 4 groups, a first group was subcutaneously injected with a phosphate buffer, and was used as control; a second group was injected with the fusion protein VF1 (dissolved in a phosphate buffer), and the injection dose was 2 mg/Kg; the injection dose of a third group was 4 mg/Kg; and the injection dose of the fourth group was 6 mg/Kg. Injection was performed two times per week. After half a month, the size of the tumor of the control group was up to 2000 mm³. All the mouse were sacrificed and anatomized, and the size of the tumor was measured, and results were shown in FIG. 4. This embodiment shows that the fusion protein VF1 has an inhibition effect on growth of tumor.

1. An anti-angiogenesis fusion protein, formed by fusing a vascular endothelial cell growth inhibitor and a variant thereof P1 with any other polypeptide P2.

2. The fusion protein according to claim 1, having a structural form of P1-P2 or P2-P1.

3. The fusion protein according to claim 1, further comprising a linker peptide.

4. The fusion protein according to claim 3, having a structural form of P1-L-P2, P2-L-P1 or P1-L-P1-L-P1.

5. The fusion protein according to claim 1, wherein the vascular endothelial cell growth inhibitor and the variant P1 are VEGI192A or a mutant thereof, and the VEGI192A or the mutant thereof has a homology with the sequence SEQ ID NO: 1 in the sequence list of 80% and more.

6. The fusion protein according to claim 3, wherein the vascular endothelial cell growth inhibitor and the variant thereof P1 is VEGI192A or a mutant thereof, and the VEGI192A or the mutant thereof has a homology with the sequence SEQ ID NO: 1 in the sequence list of 80% and more.

7. The fusion protein according to claim 1, wherein the vascular endothelial cell growth inhibitor and the variant P1 are VEGI192B or a mutant thereof, and the VEGI192B or the mutant thereof has a homology with the sequence SEQ ID NO: 2 in the sequence list of 80% and more.

8. The fusion protein according to claim 3, wherein the vascular endothelial cell growth inhibitor and the variant thereof P1 is VEGI192B or a mutant thereof, and the VEGI192B or the mutant thereof has a homology with the sequence SEQ ID NO: 2 in the sequence list of 80% and more.

9. The fusion protein according to claim 1, wherein the vascular endothelial cell growth inhibitor and the variant P1 are VEGI251 VEGI251 or a segment of VEGI251 and a mutant thereof, and the VEGI251 and the segment of VEGI251 or the mutant thereof have a homology with the sequence SEQ ID NO: 2 in the sequence list of 80% and more.

10. The fusion protein according to claim 3, wherein the vascular endothelial cell growth inhibitor and the variant thereof P1 is VEGI251 or a segment of VEGI251 and a mutant thereof, and the VEGI251 and the segment of VEGI251 or the mutant thereof have a homology with the sequence SEQ ID NO: 2 in the sequence list of 80% and more.

11. The fusion protein according to claim 1, wherein the any other polypeptide P2 is human IgG 1 Fc or a mutant thereof, and the human IgG 1 Fct or the mutant thereof has a homology with the sequence SEQ ID NO: 4 in the sequence list of 80% and more.

12. The fusion protein according to claim 3, wherein the any other polypeptide P2 is human IgG 1 Fc or a mutant thereof, and the human IgG 1 Fct or the mutant thereof has a homology with the sequence SEQ ID NO: 4 in the sequence list of 80% and more.

13. The fusion protein according to claim 3, wherein the linker peptide L is (Gly₄Ser)₃.

14. An anti-angiogenesis drug with the fusion protein according to claim 1 as active ingredient.

15. The anti-angiogenesis drug according to claim 14, wherein the anti-angiogenesis drug is used as an anti-tumor drug.

16. An anti-angiogenesis drug with the fusion protein according to claim 3 as active ingredient.

17. The anti-angiogenesis drug according to claim 16, wherein the anti-angiogenesis drug is used as an anti-tumor drug.

18. (canceled)

19. (canceled)

20. (canceled)

21. (canceled)

22. A method of preparing an anti-angiogenesis drug, the method including:
incorporating a fusion protein, formed by fusing a vascular endothelial cell growth inhibitor and a variant thereof P1 with any other polypeptide P2.

23. A method of preparing an anti-tumor drug, the method including:
incorporating a fusion protein, formed by fusing a vascular endothelial cell growth inhibitor and a variant thereof P1 with any other polypeptide P2.

24. The method according to claim 22, wherein the fusion protein further includes a linker peptide.

25. The method according to claim 23, wherein the fusion protein further includes a linker peptide.
